(19) United States
(12) Patent Application Publication
(10) Pub. No.: US 2019/0083083 A1
(43) Pub. Date: Mar. 21, 2019

(54) INTRODUCER WITH EXPANDABLE CAPABILITIES

(71) Applicant: BOSTON SCIENTIFIC SCIMED, INC., MAPLE GROVE, MN (US)

(72) Inventors: Anthony Frank Tassoni, JR., Andover, MN (US); James M. Anderson, Corcoran, MN (US); Adam David Grovender, Maple Grove, MN (US); Michael Richmon Thoreson, Maple Grove, MN (US); Scott Solberg, Ramsey, MN (US); Austin Farrell Ost, Maple Grove, MN (US); Peter John Hoffman, Brooklyn Center, MN (US); David Raab, Roseville, MN (US); Brian R. Reynolds, Ramsey, MN (US)

(73) Assignee: BOSTON SCIENTIFIC SCIMED, INC., MAPLE GROVE, MN (US)

(21) Appl. No.: 16/137,995

(22) Filed: Sep. 21, 2018

Related U.S. Application Data
(60) Provisional application No. 62/561,438, filed on Sep. 21, 2017.

(51) Int. Cl. A61B 17/02

(52) U.S. Cl. CPC A61B 17/0218; A61B 2090/3966; A61B 2017/0225

(57) ABSTRACT

An example introducer is disclosed. The example introducer includes a sheath having an inner surface and a wall having a thickness, a liner disposed along the inner surface of the sheath, the liner including at least one folded portion and a first lumen positioned in the thickness of the wall, wherein the first lumen is positioned adjacent to the at least one folded portion.

### TECHNICAL FIELD

The disclosure relates generally to medical devices and more particularly to medical devices that are adapted for use in percutaneous medical procedures.

### BACKGROUND

In some instances, performing percutaneous medical procedures may require the insertion and/or maneuvering of relatively large medical devices through a patient's vasculature. However, inserting the medical device into the vasculature may result in undesirable forces being applied to the vessel walls. For example, as the medical device passes into the vasculature, it may make undesirable contact with one or more vessel walls. This interference may cause injury to the vessel as the medical device is navigated into calcified or diseased vessels. Therefore, in some instances an introducer is utilized to facilitate the insertion of medical devices into the vessel. Further, vessel trauma resulting from forces applied to the vessel wall by a medical device may be lessened by minimizing the size of an introducer used to access the vessel. Therefore, it may be desirable to design an introducer having a reduced insertion profile, yet capable of expansion when necessary (e.g., during the passage of a medical device therethrough).

### SUMMARY

This disclosure provides design, material, manufacturing method, and use alternatives for medical devices. An example introducer includes a sheath having an inner surface and a wall having a thickness, a liner disposed along the inner surface of the sheath, the liner including at least one folded portion and a first lumen positioned in the thickness of the wall, wherein the first lumen is positioned adjacent to the at least one folded portion.

Alternatively or additionally to any of the examples above, wherein the introducer is configured to shift between a first unexpanded configuration and a second expanded configuration.

Alternatively or additionally to any of the examples above, wherein a portion of the wall of the sheath is configured to tear apart at a location adjacent the first lumen.

Alternatively or additionally to any of the examples above, wherein the tip member is designed to tear as a portion of the wall of the sheath tears apart.

Alternatively or additionally to any of the examples above, wherein the sheath includes a proximal end region, a distal end region and a length extending therebetween, and wherein the first lumen extends along the length of the sheath from the proximal end region to the distal end region.

Alternatively or additionally to any of the examples above, wherein the at least one folded portion allows the liner to radially expand.

Alternatively or additionally to any of the examples above, wherein the at least one folded portion wraps along an inner surface of the inner liner.

Alternatively or additionally to any of the examples above, wherein the sheath has a cross-sectional outer diameter, and wherein the outer diameter is substantially constant along the length of the sheath.

Alternatively or additionally to any of the examples above, wherein the liner includes a second lumen extending therein.

An introducer, comprising:

a sheath having an inner surface and a wall, the wall having a thickness; and

a liner disposed along the inner surface of the sheath, the liner including at least one folded portion;

wherein material is removed from the thickness of the wall at a position adjacent the at least one folded portion to create a preferential tear location.

Alternatively or additionally to any of the examples above, wherein material removed from the thickness of the wall defines a lumen extending within the wall thickness.

Alternatively or additionally to any of the examples above, wherein the introducer is configured to shift between a first unexpanded configuration and a second expanded configuration.

Alternatively or additionally to any of the examples above, wherein a portion of the wall of the sheath is configured to tear apart at the preferential tear location.

Alternatively or additionally to any of the examples above, wherein the tip member is designed to tear as a portion of the wall of the sheath tears apart.

Alternatively or additionally to any of the examples above, wherein the sheath includes a proximal end region, a distal end region and a length extending therebetween, and wherein the material is removed along the length of the sheath from the proximal end region to the distal end region.

Alternatively or additionally to any of the examples above, wherein the at least one folded portion allows the liner to radially expand.

Alternatively or additionally to any of the examples above, wherein the at least one folded portion wraps along an inner surface of the inner liner.

An introducer, comprising:

a hub member coupled to a tubular assembly, the tubular assembly including:
a sheath having an inner surface and a wall, the wall having a thickness;
a liner disposed along the inner surface of the sheath, the liner including at least one folded portion; and
a first lumen positioned in the thickness of the wall, wherein the first lumen is positioned adjacent to the at least one folded portion.

Alternatively or additionally to any of the examples above, wherein the introducer is configured to shift between a first unexpanded configuration and a second expanded configuration.

Alternatively or additionally to any of the examples above, wherein a portion of the wall of the sheath is configured to tear apart at a location adjacent the first lumen.

Alternatively or additionally to any of the examples above, wherein the at least one folded portion wraps along an inner surface of the inner liner.

The above summary of some examples is not intended to describe each disclosed embodiment or every implementation of the present disclosure. The Figures, and Detailed Description, which follow, more particularly exemplify these examples.

### BRIEF DESCRIPTION OF FIGURES

FIG. 1 is a perspective view of an example introducer;

FIG. 2 is a cross-sectional view of an example introducer;

FIG. 3 is a cross-sectional view of an example introducer;

FIG. 4 is a cross-sectional view of an example introducer being expanded;

FIG. 5 is a cross-sectional view of an example introducer in an expanded configuration.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some examples”, “other examples”, etc., indicate that the embodiment described may include one or more particular features, structures, and/or characteristics. However, such recitations do not necessarily mean that all examples include the particular features, structures, and/or characteristics. Additionally, when particular features, structures, and/or characteristics are described in connection with one embodiment, it should be understood that such features, structures, and/or characteristics may also be used connection with other examples whether or not explicitly described unless clearly stated to the contrary.

The following detailed description should be read with reference to the drawings in which similar elements in different drawings are numbered the same. The drawings, which are not necessarily to scale, depict illustrative examples and are not intended to limit the scope of the disclosure.

In some instances, performing percutaneous medical procedures may require the insertion and/or maneuvering of relatively large medical devices through a patient's vasculature. However, inserting the medical device into the vasculature may result in undesirable forces being applied to the vessel walls. For example, as the medical device passes into the vasculature, it may make undesirable contact with one or more vessel walls. This interference may cause injury to the vessel as the medical device is navigated into calcified or diseased vessels. Therefore, in some instances an introducer is utilize to facilitate the insertion of medical devices into the vessel. Further, vessel trauma resulting from forces applied to the vessel wall by a medical device may be lessened by minimizing the size of an introducer used to access the vessel. Therefore, it may be desirable to design an introducer having a reduced insertion profile, yet capable of expansion when necessary (e.g., during the passage of a medical device therethrough). The following examples disclose an intravascular medical device including an expandable introducer, whereby the introducer is designed to expand from a reduced profile, unexpanded configuration to an expanded configuration.

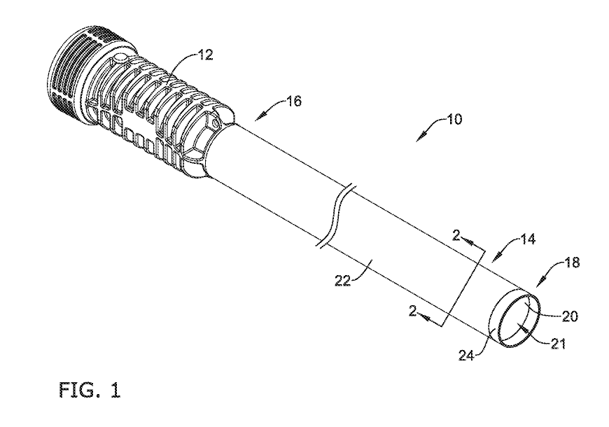

FIG. 1 illustrates an example expandable introducer (e.g., delivery sheath, access sheath, etc.) 10 including a hub member 12 coupled to a tubular assembly 14. The tubular assembly 14 may include a proximal region 16 and a distal region 18. The tubular assembly 14 may include an outer sheath 22 and an inner liner 20. The inner liner 20 may extend along an inner surface of the outer sheath 22. The tubular assembly 14 (including the sheath member 22 and liner 20) may include a lumen 21 extending therein.

While not shown in FIG. 1, the introducer 10 may include a tapered region. In some examples, the tapered region may be positioned proximal to the distal region 18 of the tubular assembly 14. In other examples, the tapered region may be positioned along the proximal region 16 of the tubular assembly 14. In some examples at least a portion of the distal region 18 of introducer assembly 14 may have a substantially constant outer diameter which transitions into a tapered portion. However, this is not intended to be limiting. It is contemplated that any portion of the introducer 10 may include any number of tapers, constant diameter regions or combinations thereof.

The hub member 12 may include a hemostatic valve or seal disposed therein. The hemostatic valve or seal may prevent blood or other bodily fluid(s) from flowing proximally through the lumen 21 of the liner 20. In at least some examples, the hub 12 may include a port in fluid communication with the lumen 21 of the liner 20.

In some examples it may be desirable to add a tip member to the distal end of any of the example introducers disclosed herein. FIG. 1 shows an example tip member 24 disposed along the distal region 18. The tip member 24 may be designed with a low durometer material. In some instances, a lower durometer material may provide the tip member 24 with the ability to radially expand (e.g., flex) outward and radially contract as a variety of medical devices are advanced through the tip member 24. Further, the tip member 24 may include a taper. While not intended to be limiting, in some examples the shape of tip member 24 may resemble a bull-nose.

Additionally, the tip member 24 may include a radiopaque material. The radiopaque material may allow the tip member 24 to be visualized by a clinician during a medical procedure. Additionally, in some examples, tip member 24 may include one or more preferential tear regions that permit the tip member 24 to tear as the introducer sheath 10 expands The tear regions of the tip member 24 may be defined as a slit, score, skive, groove, etc. which may preferentially tear upon radially expansion of tip member 24.

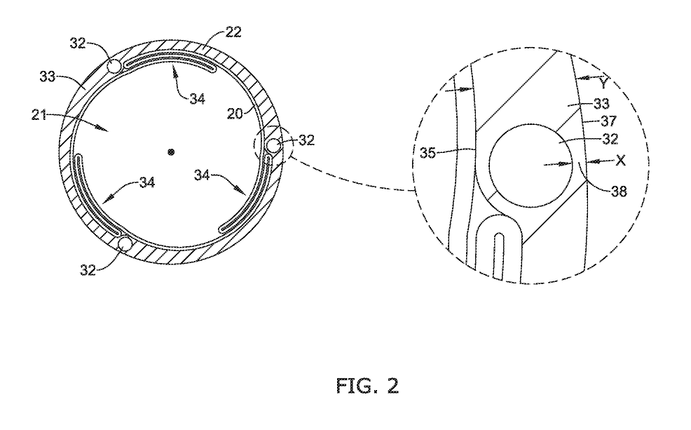

FIG. 2 shows a cross-sectional view along line 2-2 of FIG. 1. FIG. 2 illustrates the liner 20 including three folded sections 34 extending along the inner surface of the liner 20. While FIG. 2 shows three folded sections 34, it is contemplated that the liner 20 may include greater or less than three folded sections 34. For example, the liner 20 may include 1, 2, 4, 5, 6, 7, 8, 9, 10 or more folded sections 34.

It can be appreciated that FIG. 2 represents the cross-section of introducer 10 after manufacturing. For example, FIG. 2 illustrates that at least a portion of the outer sheath 22 may be engaged (e.g., attached) to at least a portion of the liner 20. For example, the inner surface of the outer sheath 22 may be engaged to at least a portion of the outer surface of the liner 20. Further, the lumen 21 may extend from a proximal end region 16 of liner 21 to a distal end region 18 of liner 20. Additionally, the lumen 21 may extend completely through an entire length of liner 20. It is contemplated that other shapes and/or configurations of the liner 20 are possible within the scope of the present disclosure.

As shown in FIG. 2, in some instances the outer sheath 22 may include one or more lumens 32 positioned at least partially within the wall thickness 33 of the outer sheath 22. The detailed view in FIG. 2 depicts the wall thickness 33 of the outer sheath as “Y.” It can be appreciated that the wall thickness “Y” may be defined as the amount of material extending from an inner surface 35 of the outer sheath 22 to the outer surface 37 of the sheath 22. Further, the detailed view of FIG. 2 illustrates an example lumen 32 positioned within the wall thickness 33 of the outer sheath 22. It can be appreciated that because the lumen 32 effectively removes material from the wall thickness 33 of the outer sheath 22, it may create a reduced wall thickness depicted as “X” within a portion of the wall thickness 33 of the outer sheath 22. As will be discussed below, this reduced wall thickness “X” may create a preferential “tear point” 38 within the wall thickness 33 of the outer sheath 22. In other words, each of the lumens 32 positioned within the wall thickness 33 of the outer sheath 22 may define a perforated tear point 38 within the wall thickness 33 of the outer sheath 22.

In at least some examples, the lumens 32 (e.g., openings, holes, spaces, etc.) or other apertures formed in the wall thickness 33 of the sheath 22 may be positioned adjacent to, radially outward of and/or directly over the one or more of the folds 34 formed in the liner 20. For example, FIG. 2 shows the lumens 32 positioned adjacent the one or more of the folds 34 in the liner 20. More particularly, in some instances the lumens 32 may be adjacent a point along the outer sheath 22 in which one or more of the folds 34 begin to wrap underneath the outer sheath 22. It can be appreciated that the point at which a lumen 32 is positioned relative to a corresponding fold 34 may be the point at which the fold 34 initially begins to expand when the liner 20 shifts from the configuration shown in FIG. 2 to an expanded configuration (as discussed in greater detail below).

As stated, the liner 20 may be described as having a compliant elongated tubular structure having a lumen 21 extending therethrough from proximal end region 16 to a distal end region 18. The liner 20 may include a wall having an inner surface and an outer surface. In some examples, a thickness of the liner wall may be defined by the inner surface and the outer surface of the liner 20.

In some examples, the liner 20 and/or the lumen 21 may be configured to radially expand from an unexpanded, delivery configuration to an expanded configuration when subjected to a radially outward force from within the lumen 21 and/or the liner 20. In at least some examples, the liner 20 may be substantially or completely compliant and/or the liner 20 may have no radial self-bias—that is, no radially inward self-bias and/or no radially outward self-bias. In other words, the liner 20 may be non-self-supporting and may not include a mechanism to radially expand and/or open on its own (i.e., absent a radially outward force exerted upon liner 20). Instead, the liner 20 may require a device or object that has a greater outer diameter than an inner diameter of the lumen 21 and/or the liner 20 to be disposed within the lumen 21 to push and/or force the liner 20 radially outward toward the expanded configuration.

Additionally, the liner 20 may not require a radially inward force be applied upon itself to collapse itself inward when there is no device, object, etc. disposed within the lumen 21. In other words, the liner 20 may not be held open or maintain a particular expanded size on its own, or liner 20 may be non-self-supporting as mentioned above. Similarly, the liner 20 may not be biased to collapse inwardly on its own. In other words, the liner 20 may take the shape and/or form of surrounding tissue(s) after being expanded. For example, a constriction in or of a vessel or body lumen in which the liner 20 is disposed may urge the liner 20 radially inward, but the liner 20 is not self-biased inwardly on its own (i.e., the liner 20 may have zero return force after expanding/opening).

As discussed above, the liner 20 may include one or more folds 34 formed therein in the delivery configuration. In at least some examples, the liner 20 may be disposed radially inward of the outer sheath 22. In some examples, the one or more folds 34 may each fold back on themselves to form, for example, a wave shape, an S-shape, T-shape and/or a Z-shape when viewed in cross-section. In some examples, each of the one or more folds forming a T-shape may include two or more distinct waves, S-shaped, or Z-shaped secondary folds within and thus forming each of the one or more T-shaped folds. Other shapes and configurations, while not expressly illustrated, are also contemplated.

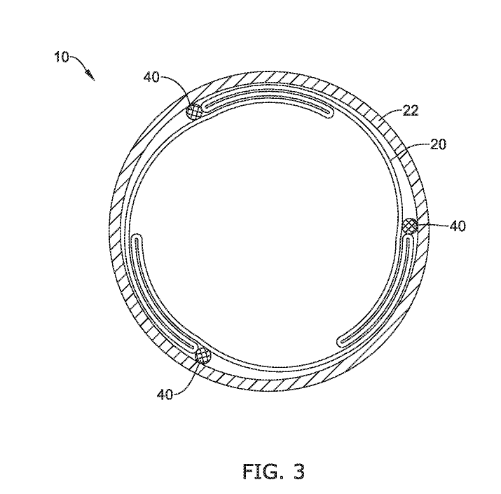

FIG. 3 illustrates one example methodology to construct the introducer 10 (shown in FIG. 1 and FIG. 2). Example manufacturing steps may include configuring the outer sheath 22 and the liner 20 as shown in FIG. 3. Additionally, to form the lumens 32 (shown in FIG. 2), mandrels 40 may be positioned as shown in FIG. 3. In other words, the mandrels 40 (which are the desired shape of the lumen 32), may be positioned between the outer sheath 22 and the liner 20. After configuring the outer sheath 22, the liner 20 and the mandrels 40 as shown in FIG. 3, heat may be applied such that outer sheath member 22 melts (e.g., reflows) and engages with the liner 20. In some examples, the outer sheath 22 may be fixedly attached to the outer surface of the liner 20. In some examples, the outer sheath 22 may be bonded, laminated, fused, glued, co-molded, melted, welded, or other suitable means to the liner 20. In other words, in some examples, the outer sheath 22 may be permanently attached to the outer surface of the liner 20. Further, upon completion of the melting (e.g., reflow) process, the mandrels 40 may be removed to create the lumens 32 shown in FIG. 2.

In some examples, the outer sheath 22 may be formed from the same material as the liner 20. In some examples, the outer sheath 22 may be formed from a different material than the liner 20. In some examples, some or all of the outer sheath 22 may be fixedly attached to the outer surface of the wall of the liner 20. In some examples, the entire outer sheath 22 may be fixedly attached to the outer surface of the wall of the liner 20.

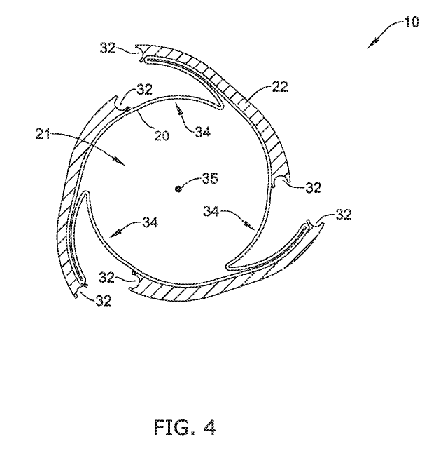

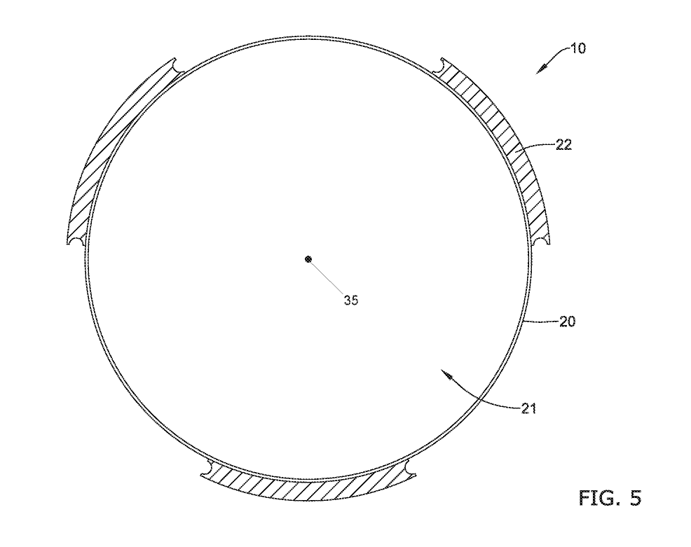

In some examples the liner 20 may be configured to permit the lumen 21 to radially expand from a first inner diameter to a second inner diameter. For example, FIG. 4 and FIG. 5 illustrates the introducer 10 expanding from a first unexpanded configuration to a second expanded configuration.

In some examples, the outer sheath 22 may be configured to separate, split, or tear as the liner 20 is expanded from a first configuration to a second, expanded configuration. The expansion may be due to a radially outward force applied from within the liner 20. In some examples, the outer sheath 22 may be configured to separate, split, perforate and/or tear along and/or through the plurality of the lumens 32 (e.g., along the tear points 38 discussed above with respect to FIG. 2), or other weakening features formed in the wall of the outer sheath 22. In other words, the outer sheath 22 may separate, split, or tear where the outer sheath 22 is discontinuous and/or constructed of a thinner material (e.g., along tear points 38). As discussed above, as the liner 20 radially expands outward toward an expanded configuration, the outer sheath 22 may tear along preferential tear points corresponding to the lumens 32. Further, the expansion of the sheath 22 may correlate with the expansion of the lumen 21. The lumen 21 may expand from a first inner diameter to a second inner diameter greater than the first inner diameter.

As discussed above, the plurality of lumens 32, or other weakening features formed in the wall of the outer sheath 22 may be positioned adjacent to, radially outward of, in communication with, and/or directly over the one or more folds 34 formed in the liner 20. As mentioned above, in some examples, the plurality of lumens 32, or other weakening features formed in the wall of the outer sheath 22 may be arranged in one or more longitudinal lines along the length of the expandable introducer 10, the liner 20 and/or the outer sheath 22. Accordingly, in some examples, the plurality of lumens 32 or other weakening features formed in the wall of the outer sheath 22 may be arranged in one or more longitudinal lines adjacent to, radially outward of, in communication with, and/or directly over the one or more folds 34 and/or the two or more secondary folds 34 formed in the liner 20. In some examples, the one or more longitudinal lines may directly correspond to the one or more folds 34 and/or the two or more secondary folds 34. In some examples, the reduced thickness region and/or a substantially thinner thickness of the outer sheath 22 may be disposed adjacent to, radially outward of, in communication with, and/or directly over the one or more folds 34 and/or the two or more secondary folds 34 of the liner 20.

FIG. 5 illustrates the introducer 10 after having been expanded to an expanded configuration. As shown in FIG. 5, the liner 20 has been expanded to an expanded configuration. FIG. 5 may represent the configuration of the introducer 10 prior to the insertion of additional example medical device therethrough.

It is contemplated that the liner 20 may be configured to substantially prevent axial stretching along the lumen 21. In other words, the liner 20 may permit the lumen 21 to expand radially outward from a central longitudinal axis 35 of the liner 20 and/or the expandable introducer 10 without stretching or expanding in an axial or longitudinal direction. In some examples, the second inner diameter may be greater than the first outer diameter. Because the liner 20 may be made from an inelastic material, it may be configured to expand radially outward to a predetermined maximum second inner diameter, but may not stretch or expand radially outward beyond the predetermined maximum second inner diameter.

In some examples, the inner surface of the wall of the liner 20 may include one or more layers or coatings, such as a lubricious coating, a hydrophilic coating, a hydrophobic coating, or other suitable coatings, and the like, or the liner 20 may include a lubricant disposed within the lumen 21. In some examples, an outer surface of the expandable introducer 10 and/or the outer sheath 22 may include one or more layers or coatings, such as a lubricious coating, a hydrophilic coating, a hydrophobic coating, or other suitable coating, and the like, or the expandable introducer 10 and/or the outer sheath 22 may include a lubricant disposed upon the outer surface thereof.

In some examples, the example expandable introducer 10 may be disposed about or inserted over a guidewire (not shown), although the guidewire is not required. In some examples, the expandable introducer 10 and/or the liner 20 may include a proximal non-expandable section and a distal expandable section. In examples having a proximal non-expandable section, the proximal non-expandable section may have an inner diameter or extent sufficient to accept a medical device passing therethrough, while the distal expandable section may have an inner diameter or radial extent in a relaxed condition that is less than a maximum outer diameter or extent of the medical device. The expandable introducer 10 and/or the liner 20 may be formed using any of the techniques or structures discussed herein.

A method of use of the introducer 10 may include inserting the introducer 10 into and/or navigated within a vessel or body lumen to a target site or area of interest. In some embodiments, the vessel or body lumen may include a partial or total occlusion or obstruction formed therein. Importantly, the introducer 10 may be used in a vessel or body lumen that does not include a partial or total occlusion therein.

After navigating the introducer 10 to the target site or area of interest, an example medical device may be inserted through the hub member 12 and into the lumen 21 of the introducer 10. As the example medical device reaches, encounters, and/or engages the lumen 21 of the liner 20, the medical device may exert a radially outward force from within the lumen 21 upon the wall of the liner 20 and/or the outer sheath 22. The radially outward force may cause the outer sheath 22 to separate, split, or tear along and/or through the plurality of preferential tear points, or other weakening features, formed in the wall of the outer sheath 22 as the example medical device is advanced distally through the lumen 21 of the liner 20. As the outer sheath 22 separates, splits, or tears apart, the one or more folds 34 of the liner 20 are permitted to unfold and increase the inner diameter of the lumen 21. In a vessel or body lumen having a partial or total occlusion, when the dilator may be advanced through the occlusion and/or the outer sheath 22 is opened/expanded within the occlusion, the vessel or body lumen may be deformed or distended by the rigidity of the medical device moving the occlusive material radially outward.

In some examples, introducer 10 (and/or components thereof) may be made from materials such as metals, metal alloys, polymers, ceramics, metal-polymer composites, or other suitable materials, and the like. Some examples of suitable materials may include metallic materials such as stainless steels (e.g. 304v stainless steel or 316L stainless steel), nickel-titanium alloys (e.g., nitinol, such as super elastic or linear elastic nitinol), nickel-chromium alloys, nickel-chromium-iron alloys, cobalt alloys, nickel, titanium, platinum, or alternatively, a polymeric material, such as a high performance polymer, or other suitable materials, and the like. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

In some examples, the introducer 10 (and/or components thereof) may be made from materials such as, for example, a polymeric material, a ceramic, a metal, a metal alloy, a metal-polymer composite, or the like. Examples of suitable polymers may include polyurethane, a polyether-ester such as ARNITEL® available from DSM Engineering Plastics, a polyester such as HYTREL® available from DuPont, a linear low density polyethylene such as REXELL®, a polyamide such as DURETHAN® available from Bayer or CRISTAMID® available from Elf Atochem, an elastomeric polyamide, a block polyamide/ether, a polyether block amide such as PEBA available under the trade name PEBAX®, silicones, polyethylene, Marlex high-density polyethylene, polyetheretherketone (PEEK), polyimide (PI), and polyetherimide (PEI), a liquid crystal polymer (LCP) alone or blended with other materials. In some examples, a suitable polymeric material may have a yield strain of at least 20%, at least 30%, at least 40%, at least 50%, or more. In some examples, the sheath, the membrane, and/or the plurality of corrugations may be made from a material having a low coefficient of friction. In some examples, the sheath, the membrane, and/or the plurality of corrugations may be formed from a fluoropolymer, such as polytetrafluoroethylene (PTFE) or fluorinated ethylene propylene (FEP).

Portions of introducer 10 (and/or components thereof) may be made of, may be doped with, may include a layer of, or otherwise may include a radiopaque material. Radiopaque materials are understood to be materials capable of producing a relatively bright image on a fluoroscopy screen or another imaging technique such as X-ray during a medical procedure. This relatively bright image aids the user of device in determining its location. For example, one or more of the elements described above (i.e., the sheath, the membrane, the medical device, etc.) may include or be formed from a radiopaque material. Suitable materials can include, but are not limited to, bismuth subcarbonate, iodine, gold, platinum, palladium, tantalum, tungsten or tungsten alloy, and the like.

It should be understood that although the above discussion was focused on percutaneous medical procedures within the vasculature of a patient, other examples or methods in accordance with the disclosure can be adapted and configured for use in other parts of the anatomy of a patient. For example, devices and methods in accordance with the disclosure can be adapted for use in the digestive or gastrointestinal tract, such as in the mouth, throat, small and large intestine, colon, rectum, and the like. For another example, devices and methods can be adapted and configured for use within the respiratory tract, such as in the mouth, nose, throat, bronchial passages, nasal passages, lungs, and the like. Similarly, the devices and methods described herein with respect to percutaneous deployment may be used in other types of surgical procedures as appropriate. For example, in some examples, the devices may be deployed in a non-percutaneous procedure. Devices and methods in accordance with the disclosure can also be adapted and configured for other uses within the anatomy.

It should be understood that this disclosure is, in many respects, only illustrative. Changes may be made in details, particularly in matters of shape, size, and arrangement of steps without exceeding the scope of the disclosure. The disclosure's scope is, of course, defined in the language in which the appended claims are expressed.

What is claimed is:

1. An introducer, comprising:
a sheath having an inner surface and a wall, the wall having a thickness;
a liner disposed along the inner surface of the sheath, the liner including at least one folded portion; and
a first lumen positioned in the thickness of the wall, wherein the first lumen is positioned adjacent to the at least one folded portion.

2. The introducer of claim 1, wherein the introducer is configured to shift between a first unexpanded configuration and a second expanded configuration.

3. The introducer of claim 1, wherein a portion of the wall of the sheath is configured to tear apart at a location adjacent the first lumen.

4. The introducer of claim 3, wherein the tip member is designed to tear as a portion of the wall of the sheath tears apart.

5. The introducer of claim 1, wherein the sheath includes a proximal end region, a distal end region and a length extending therebetween, and wherein the first lumen extends along the length of the sheath from the proximal end region to the distal end region.

6. The introducer of claim 1, wherein the at least one folded portion allows the liner to radially expand.

7. The introducer of claim 1, wherein the at least one folded portion wraps along an inner surface of the inner liner.

8. The introducer of claim 1, wherein the liner includes a second lumen extending therein.

9. An introducer, comprising:
a sheath having an inner surface and a wall, the wall having a thickness; and
a liner disposed along the inner surface of the sheath, the liner including at least one folded portion;
wherein material is removed from the thickness of the wall at a position adjacent the at least one folded portion to create a preferential tear location.

10. The introducer of claim 9, wherein material removed from the thickness of the wall defines a lumen extending within the wall thickness.

11. The introducer of claim 9, wherein the introducer is configured to shift between a first unexpanded configuration and a second expanded configuration.

12. The introducer of claim 9, wherein a portion of the wall of the sheath is configured to tear apart at the preferential tear location.

13. The introducer of claim 9, wherein the tip member is designed to tear as a portion of the wall of the sheath tears apart.

14. The introducer of claim 9, wherein the sheath includes a proximal end region, a distal end region and a length extending therebetween, and wherein the material is removed along the length of the sheath from the proximal end region to the distal end region.

15. The introducer of claim 9, wherein the at least one folded portion allows the liner to radially expand.

16. The introducer of claim 9, wherein the at least one folded portion wraps along an inner surface of the inner liner.

17. An introducer, comprising:
a hub member coupled to a tubular assembly, the tubular assembly including:
a sheath having an inner surface and a wall, the wall having a thickness;
a liner disposed along the inner surface of the sheath, the liner including at least one folded portion; and
a first lumen positioned in the thickness of the wall, wherein the first lumen is positioned adjacent to the at least one folded portion.

18. The introducer of claim 17, wherein the introducer is configured to shift between a first unexpanded configuration and a second expanded configuration.

19. The introducer of claim 17, wherein a portion of the wall of the sheath is configured to tear apart at a location adjacent the first lumen.

20. The introducer of claim 17, wherein the at least one folded portion wraps along an inner surface of the inner liner.
